(19) United States
(12) Patent Application Publication
(10) Pub. No.: US 2019/0000601 A1
(43) Pub. Date: Jan. 3, 2019

(54) Bristle-Less Teeth Cleaning Device with Automatic Gel Dispensing for Combined Mechanical and Chemical Activation

(71) Applicants: Chao Huang, Syracuse, NY (US); Tairan Li, Syracuse, NY (US)

(72) Inventors: Chao Huang, Syracuse, NY (US); Tairan Li, Syracuse, NY (US)

(21) Appl. No.: 16/024,056

(22) Filed: Jun. 29, 2018

Related U.S. Application Data
(60) Provisional application No. 62/526,497, filed on Jun. 29, 2017.

(51) Int. Cl. A61C 17/22; A61C 17/34; A61C 17/022; A61C 19/06; A61N 5/06; A61C 1/00

(52) U.S. Cl. CPC A61C 17/228; A61C 17/3481; A61C 17/022; A61C 19/066; A61C 17/224; A61N 2005/0652; A61C 17/221; A61C 1/0015; A61C 2204/002; A61N 2005/0606; A61N 2005/0626; A61N 5/062

(57) ABSTRACT

Provided is a teeth cleaning device that includes a gel cartridge, a plurality of light emitting diodes, a mouth guard, and a controller. The cartridge stores a gel. The light emitting diodes emit a cold light. The mouth guard has a generally arcuate shape and includes a first recess, a second recess opposite the first recess, a cavity, a first surface, and a plurality of first openings. The first recess is located on a top side of the guard to receive a first row of teeth, and the second recess is located on the bottom side of the guard to receive a second row of teeth. The cavity is between the first recess and the second recess. The first surface is in communication with the cold light of the light emitting diodes. The assembly has a generally arcuate shape and is disposed in the cavity of the guard between the first recess and the second recess. The assembly includes a plurality of vibration motors and a plurality of second openings, wherein the motors are configured to generate vibrations and the second openings are in communication with the first openings. The controller is configured to control dispensing the gel from the gel cartridge through the second openings and the first openings into a user's oral cavity, emitting the cold light from the light emitting diodes through the mouth guard into the user's oral cavity to activate a whitening compound in the gel, and vibrating the plurality of vibration motors configured to drive the gel against surfaces of the first row of teeth and the second row of teeth.

### CROSS-REFERENCE TO RELATED APPLICATIONS

This application claims benefit of U.S. Provisional Patent Application No. 62/526,497, filed on Jun. 29, 2017, the contents of which are incorporated herein by reference in their entirety.

### BACKGROUND

### Field

The present application relates to devices for cleaning teeth. More specifically, the present application is directed to a teeth cleaning device that is provided with automatic gel dispensing for combined mechanical and chemical activation, improving the cleaning and/or whitening of the teeth.

### Brief Discussion of Related Art

Dental health and oral hygiene are significantly important to personal health. Clean and healthy teeth not only keep a person otherwise healthy, but the appearance of clean and healthy teeth improves a person's level of confidence, which improves the person's health both on a physical level as well as psychological level.

Cleaning teeth removes plaque, reduces decay, and also helps to decrease the chance of getting oral diseases. In the marketplace, there are various teeth/mouth cleaning devices. Of the various existing devices, the manual toothbrush is conventional and the most universally recognized teeth cleaning device. The toothbrush is conventionally used in combination with toothpaste in order to clean the teeth and also partly the gums. The toothbrush typically includes a handle, head, and bristles. The bristles are embedded in the head and the head is supported at one end of the handle. A user grasps the handle and maneuvers the handle to insert the head into the mouth so that the bristles touch the teeth, and moves the handles in a repetitive pattern, e.g., up-and-down, circular, and/or another pattern, across the teeth until all teeth are cleaned.

Manual brushing of teeth requires that a user devote considerable attention to the proper pattern or technique. On the one hand, incorrect manual brushing skills can lead to vulnerable teeth by missing out important regions of teeth that can decay as a result. On the other hand, incorrect manual brushing skills can also lead to abrasion, which wears off the protective enamel of the teeth that can also result in the decay of teeth. Moreover, certain users, whether as a result of neglect or busy schedules, fail to brush long enough in order to clean teeth completely and reduce incidence of decay.

The development of motorized toothbrushes has added convenience as well as enhanced effectiveness to the teeth brushing process. While a motorized toothbrush rotates and/or vibrates the bristles, the motorized toothbrush also requires user's manual movement of the handle so as to guide the head with the bristles along the teeth until all teeth are cleaned.

Some other motorized teeth cleaning devices include a u-shaped tray. The tray is typically filled with a large number of bristles that surround a row of teeth in the user's mouth. A handle that extends to the exterior of the mouth (external handle) is attached to the tray, and includes a motor housing within which a motor is housed in order to vibrate the tray, and accordingly the bristles that surround the teeth. The brush tray is capable of receiving the user's row of teeth when the user inserts their top row or bottom row of teeth into the tray using the external handle. Typically, the user manually dispenses toothpaste into the tray.

While the motorized u-shaped tray device does not require the user's manual movement of the handle as do manual or motorized toothbrushes, the motorized u-shaped tray device does however require that the user hold the external handle while the device is in use. More specifically, the user must hold the external handle so as to keep the tray level for the proper degree and uniformity of pressure across the row of teeth. Moreover, the user must also hold the external handle in order to switch between cleaning a top row of teeth and a bottom row of teeth in the user's mouth. A user who does not appropriately hold and manipulate the external handle, would not receive the required degree and uniformity of pressure required for cleaning, and would not receive cleaning of both rows of teeth.

In the foregoing regard, if a user wished to let go the external handle, the user would have to clench their jaws and apply a significant biting pressure to hold the tray in place in order to counterbalance the weight of the external handle. Such biting pressure can compromise the appropriate degree and uniformity of pressure applied to the user's teeth, and can further pose the risk of damaging the user's teeth and/or jaws.

A variety of toothpaste dispensing devices and teeth whitening devices exist, but they have not been combined into an ergonomic form factor that is esthetic and yet provides sufficient bristle-free operation for combined cleaning and/or whitening teeth.

It is therefore desirable to provide lightweight bristle-less teeth cleaning device that can automatically dispense a gel (e.g., toothpaste or the like) into a u-shaped tray in order to provide combined mechanical and chemical activation of the gel for cleaning and/or whitening of the teeth.

### SUMMARY

In accordance with an embodiment, a bristle-less teeth cleaning device that provides combined mechanical and chemical activation for improved cleaning and/or whitening of teeth is disclosed.

The teeth cleaning device includes a gel cartridge, a plurality of light emitting diodes, a mouth guard, and a central controller. The gel cartridge is configured to store a cleaning and whitening gel. Moreover, the light emitting diodes are configured to emit a cold light.

The mouth guard has a generally arcuate shape and includes a first arcuate recess, a second arcuate recess opposite the first arcuate recess, a cavity, a first surface, and a plurality of first openings. The first arcuate recess is located on a top side of the mouth guard to receive a first row of teeth, while the second arcuate recess is located on the bottom side of the mouth guard to receive a second row of teeth. The cavity is located between the first recess and the second recess. Moreover, the first surface is in communication with the cold light of the light emitting diodes. In some cases, the mouth guard can be transparent.

The vibration and dispensing assembly also has a generally arcuate shape and is disposed in the cavity of the mouth guard between the first arcuate recess and the second arcuate recess. The assembly includes a plurality of vibration motors and a plurality of second openings. The motors are configured to generate vibrations. The second openings are in communication with the first openings.

The central controller configured to control dispensing the gel from the gel cartridge through the second openings and the first openings into a user's oral cavity, emitting the cold light from the light emitting diodes through the mouth guard into the user's oral cavity to activate a whitening compound in the gel, and vibrating the plurality vibration motors configured to drive the gel against surfaces of the first row of teeth and the second row of teeth.

In some cases, the teeth cleaning device can include an outer shell that has one or more removable shell sections, wherein the outer shell is configured to house the gel cartridge, the light emitting diodes, the vibration and dispensing assembly, and the controller. Moreover, the outer shell can include a main shell section having a cavity. The main shell section can have a generally circular opening terminating with a planar bottom surface on a first side, and a generally oval opening at a top of a second side, wherein the generally oval opening is in communication with the circular opening.

In some cases, the teeth cleaning device can further include air tubes, gel tubes, and an air pump that is configured to pressurize the gel cartridge using air tubes, and dispense the gel from the gel cartridge using gel tubes. Moreover, the air pump can be disposed in the main shell section and the gel tubes engage gel guides of the vibration and dispensing assembly through the oval opening.

In some cases, the main shell section can also include an air vent connected by a first air tube to an input of the air pump in order to receive ambient air. A second air tube can be connected from an output of the air pump to an input in the gel cartridge. Moreover, a gel tube can be connected to an output of the cartridge.

In some cases, the light emitting diodes are disposed in the main shell section and emit the cold light through the oval opening into the first surface of the mouth guard.

In some cases, the teeth cleaning device can include a cold light controller that is configured to communicate with the central controller to control the emitting of the cold light from the cold light emitting diodes. Moreover, the cold light emitting diodes can be disposed on the cold light controller.

In some cases, the teeth cleaning device can include a rechargeable battery, and a charging controller configured to provide power from the rechargeable battery to the central controller. Moreover, the teeth cleaning device can also include an induction coil configured to generate power when in proximity to a second induction coil, and the charging controller is configured to charge the rechargeable battery.

In some cases, the teeth cleaning device can include a plurality of color light emitting diodes, wherein the central controller is configured to control the color generating light emitting diodes to emit color light indicating at least one operational status of the teeth cleaning device. Moreover, a light strip can be disposed flush between several shell sections of an outer shell, and the plurality of color light emitting diodes can be disposed on or embedded in the light strip. The light strip can be transparent.

In some cases, the vibration and dispensing assembly can include a sensor configured to detect pressure from a user's bite through the mouth guard. Moreover, the central controller can be configured to control dispensing the gel, emitting the cold light, and vibrating the motors based on the pressure detected by the pressure sensor.

In some cases, the central controller can be configured to control dispensing the gel for a first predetermined period of time, then followed by emitting the cold light and vibrating the motors for a second predetermined period of time. The first predetermined period of time can be three seconds, and the second predetermined period of time can be ten seconds. Moreover, the central controller can adjust a pattern or a frequency of vibrating the motors based on an amount of the pressure detected by the pressure sensor.

These and other purposes, goals and advantages of the present application will become apparent from the following detailed description of example embodiments read in connection with the accompanying drawings.

### BRIEF DESCRIPTION OF THE DRAWINGS

Some embodiments are illustrated by way of example and not limitation in the figures of the accompanying drawings in which:

FIGS. 1A-1D illustrate an assembled bristle-less teeth cleaning device that can automatically dispense a cleaning and whitening gel in order to provide a combined mechanical and chemical activation of the gel for cleaning and whitening of a user's teeth;

FIG. 2 illustrates an exploded view of example assemblies included in the bristle-less teeth cleaning device illustrated in FIG. 1;

FIGS. 3A and 3B illustrate an example mouth guard illustrated in FIGS. 1 and 2;

FIGS. 4A-4C illustrate an example vibration and dispensing assembly 202 illustrated in FIG. 2;

FIGS. 5A-5D illustrate a light assembly and example integration of the light assembly into a main shell section illustrated in FIG. 2;

FIGS. 6A-6D illustrate a gel assembly and example integration of the gel assembly into the main shell section illustrated in FIG. 2;

FIGS. 7A-7C illustrate a charging assembly and a central controller, and their example integration into the main shell section illustrated in FIG. 2;

FIG. 8 illustrates the teeth cleaning device of FIG. 1 with shell sections, light strip, and mouth guard removed, exposing the assembly and integration of electrical and/or electronic components into the main shell section of the teeth cleaning device illustrated in FIGS. 1 and 2;

FIG. 9 is a circuit diagram that illustrates example electrical connections among the electrical and/or electronic components of the teeth cleaning device illustrated in FIG. 1;

FIG. 10 illustrates an example charging/cleaning device for the teeth cleaning device illustrated in FIGS. 1-9;

FIGS. 11A-11C illustrate several views of the example charging/cleaning device illustrated in FIG. 10 with the example teeth cleaning device illustrated in FIGS. 1-9; and

FIG. 12 illustrates a logical diagram of the teeth cleaning device illustrated in FIGS. 1-9, as well as the charging/cleaning device illustrated in FIGS. 10 and 11.

### DETAILED DESCRIPTION

A lightweight bristle-less teeth cleaning device that can automatically dispense toothpaste into a u-shaped tray in order to provide combined mechanical and chemical activation of the toothpaste for teeth cleaning and/or whitening is disclosed herein. In the following description, for the purposes of explanation, numerous specific details are set forth in order to provide a thorough understanding of example embodiments. It will be evident, however, to one skilled in the art, that an example embodiment may be practiced without all of the disclosed specific details.

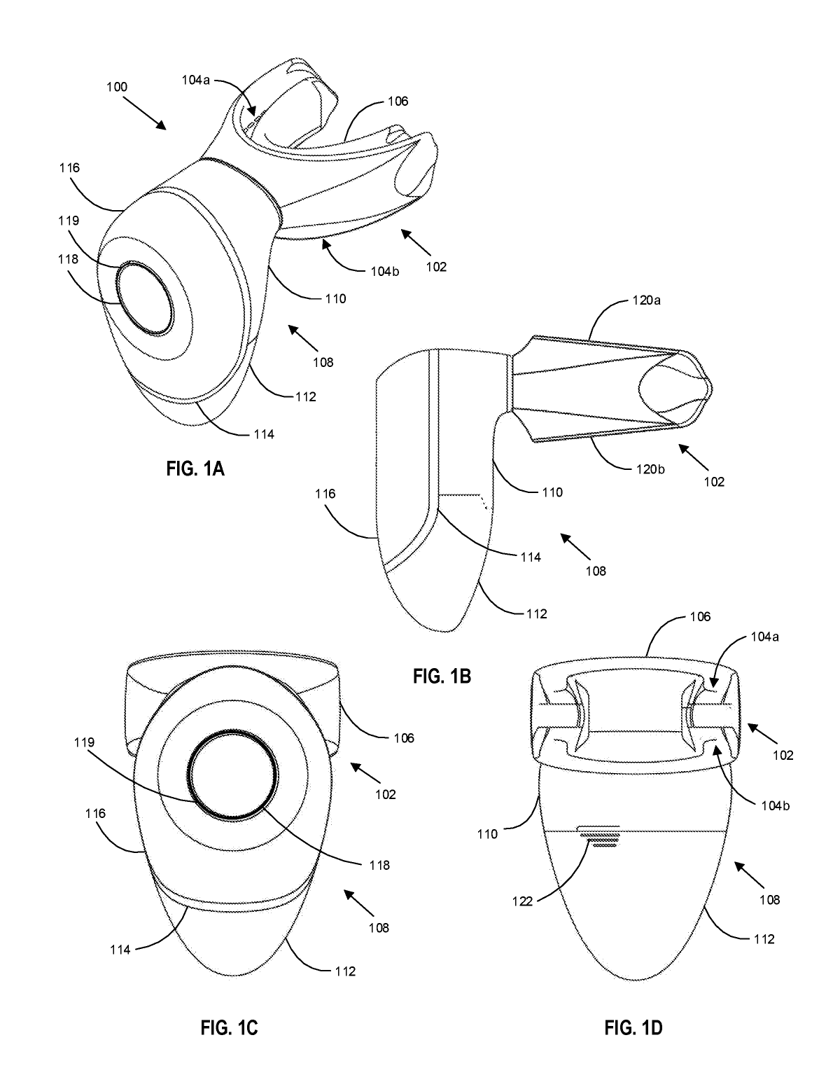

FIGS. 1A-1D illustrate an assembled bristle-less teeth cleaning device 100 that can automatically dispense cleaning and whitening gel in order to provide a combined mechanical and chemical activation of the gel for cleaning and whitening of a user's teeth. The teeth cleaning device 100 includes a mouth assembly 102 and a shell assembly 108.

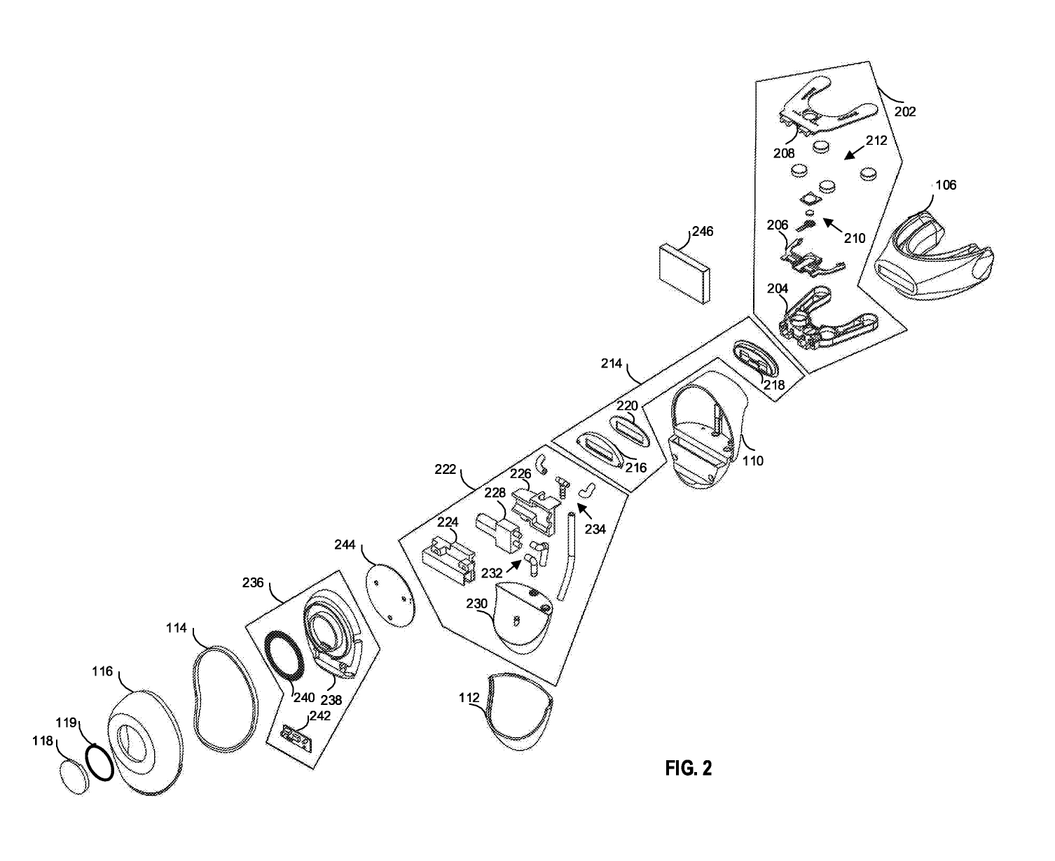

The mouth assembly 102 includes a mouth guard 106, as well as a vibration and dispensing assembly 202, as described in greater detail hereinafter with reference to FIG. 2. The mouth guard 106 is configured to fit into a user's mouth and contact the user's teeth, e.g., the top row of teeth and the bottom row of teeth. The mouth guard 106 can be a one-size fits all, or can be provided in multiple sizes for different users. Moreover, the mouth guard 106 can be customized for certain users in order to make sure that the mouth guard 106 can closely match the user's oral cavities as well as structure and/or location of the user's teeth.

In the foregoing regard, the mouth guard 106 is made of a polyurethane elastomer so that mouth guard 106 can deform sufficiently in the user's mouth in order to fit precisely over the user's teeth, and can thus conform to various oral cavities, teeth structures and/or locations of teeth in various users.

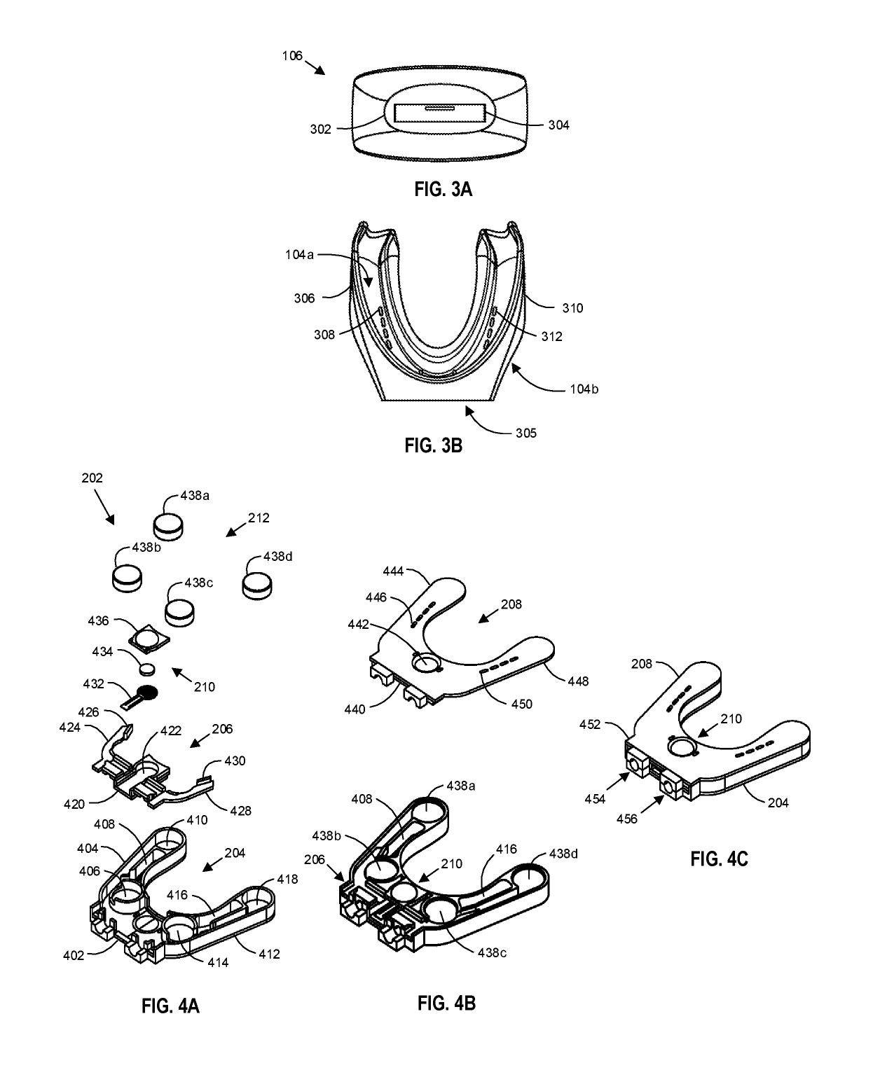

As particularly illustrated in FIGS. 1A and 1D, the mouth guard 106 includes an arcuate recess 104a in the top of the mouth guard, and an arcuate recess 104b in the bottom of the mouth guard, in order to receive the user's top and bottom rows of teeth, respectively. As particularly illustrated in FIG. 1B, the mouth guard 106 has a generally sloped side profile that extends from the shell assembly 108 toward the end of the mouth guard 106. The sloped profile is configured to precisely fit the user's oral cavity, cover the top and bottom rows of teeth as well as gums, and enable the user to bite down on the mouth guard 106 for certain activations of the teeth cleaning device as will be described hereinafter in great detail. The mouth guard 106 is described in greater detail hereinafter in reference to FIGS. 3A and 3B.

The shell assembly 108 is configured to engage the mouth assembly 102 in a water tight engagement. Moreover, the shell assembly 108 is configured to house various electrical, mechanical, and replaceable and/or fungible components of the teeth cleaning device 100, as will be described in detail hereinbelow with reference to FIG. 2. The shell assembly 108 is made of plastic, such as acrylonitrile butadiene styrene (ABS), another plastic, or a combination of various plastics. The one or more plastics that can be used can include polyamides (PA), polycarbonate (PC), polyester (PES), polyethylene (PE), high-density polyethylene (HDPE). low-density polyethylene (LDPE), polyethylene terephthalate (PET), polypropylene (PP), polystyrene (PS), high impact polystyrene (HIPS), polyurethanes (PU), polyvinyl chloride (PVC), polyvinylidene chloride (PVDC), acrylonitrile butadiene styrene (ABS), acrylonitrile butadiene styrene (ABS), or one or more combinations thereof.

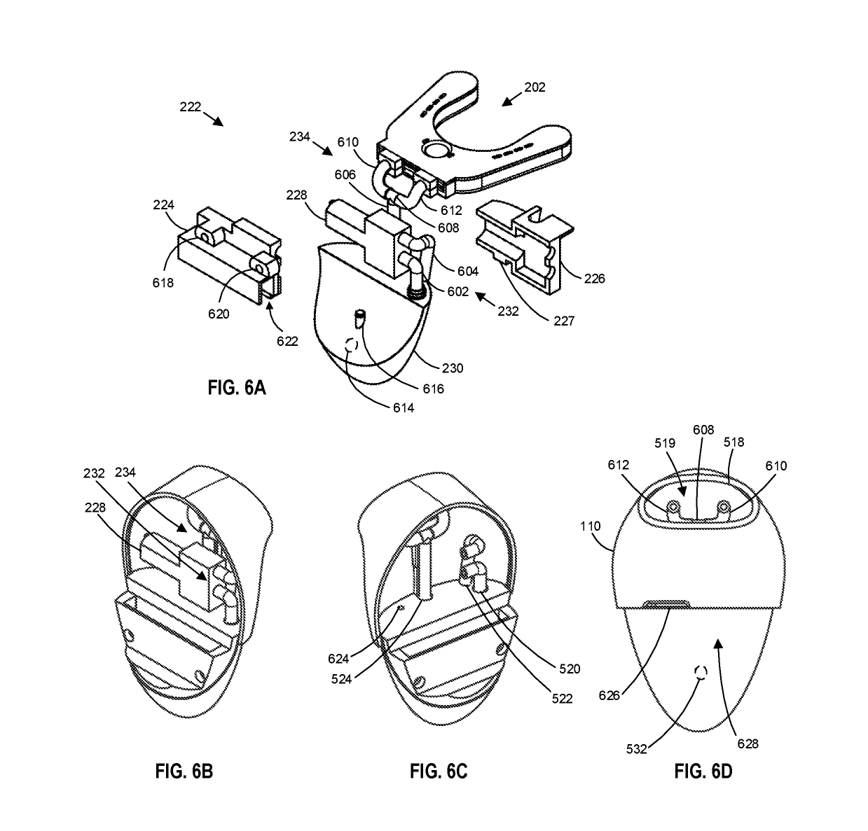

The shell assembly 108 forms an outer shell of the teeth cleaning device 100. In this regard, the shell assembly 108 includes removable shell sections 110-118, as well as a light strip 119. The shell sections 110-118 can be snap-fit together to provide an ergonomic form factor, which can also be opened in order to replace one or more of the components of the teeth cleaning device 100, as will be described in greater detail with reference to FIGS. 6A-6D. The light strip 119 is generally circular and is disposed between the shell sections 116, 118. Moreover, the light strip 119 is configured to indicate one or more operational statuses of the teeth cleaning device 100, as will be described in greater detail with reference to FIGS. 2, 9, and 12.

Shell section 110 is a main section of the shell assembly 108 that will receive and engage various electrical, mechanical, and replaceable and/or fungible components of the teeth cleaning device 100, as will be described in detail hereinbelow with reference to FIG. 2.

The shell section 112 serves as a removable section for users to be able to replace and/or refill a gel cartridge 230 of the teeth cleaning device 100, which is described in greater detail hereinbelow with reference to FIGS. 2 and 6A-6D. The shell section 112 can be snap-fit into engaged position with the shell sections 110, 114, and 116, and can further be removed therefrom for replacement/refill of the gel cartridge 230. As particularly illustrated in FIG. 1D, the shell section 112 includes a finger grip 122 to facilitate removal of the shell section 112 from the teeth cleaning device 100.

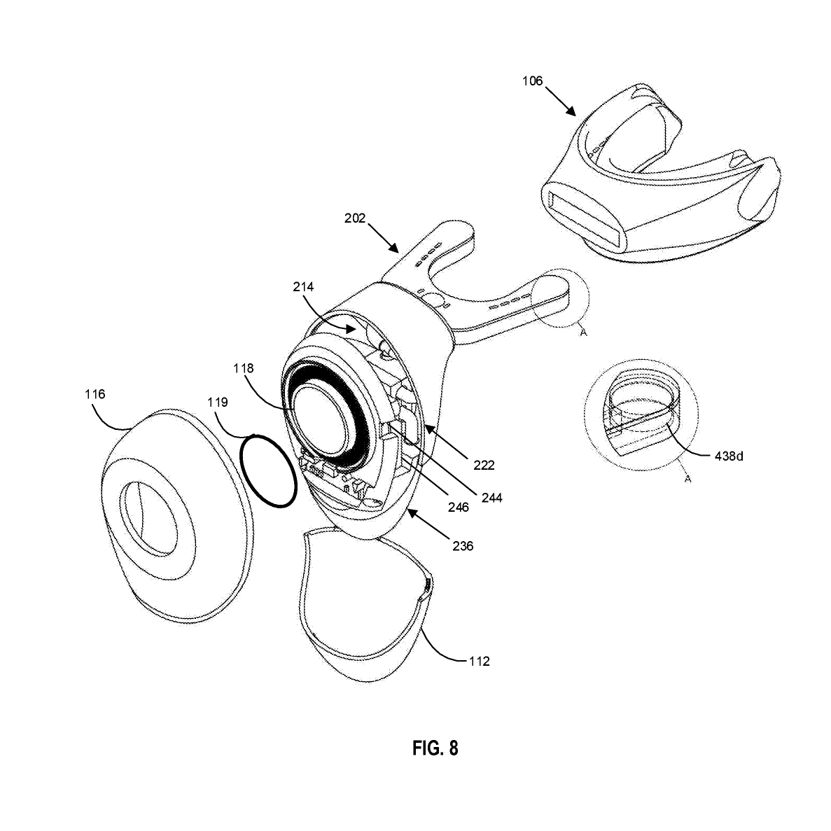

The shell section 114 is a decor ring that is disposed between the shell sections 110, 112, and 116. The shell section 116 is a front shell section of the teeth cleaning device 110, while the shell section 118 is a cover plate that is received flush in an opening in the shell section 116, and engages a housing 238 of a charging assembly 236, as illustrated in greater detail in FIGS. 2 and 8. The light strip 119 is disposed flush between the shell sections 116, 118.

FIG. 2 illustrates an exploded view of example assemblies included in the bristle-less teeth cleaning device 100 illustrated in FIG. 1. The teeth cleaning device 100 includes a vibration and dispensing assembly 202, a light assembly 214, a gel assembly 222, a charging assembly 236, a mouth guard 106 of the mouth assembly 102, and shell sections 110-118 of the shell assembly 108.

The vibration and dispensing assembly 202 has a generally arcuate shape and is configured to activate dispensing of gel and activate vibration, e.g., provide start/stop of operation, as well as provide various patterns and/or frequencies of vibration. More specifically, the vibration and dispensing assembly 202 can dispense the cleaning and whitening gel into the user's mouth through the mouth guard 106, and can provide one or more patterns of vibration in order to distribute the gel about the surfaces of the teeth, allowing the mouth guard 106 to gently vibrate the gel against the surfaces of the teeth and gums, helping to remove dental plaque and/or tartar buildup on the user's teeth, while also helping to massage the user's gums. The patterns or cadence of activation are described in greater detail hereinbelow.

As the mouth guard 106 fits precisely into the user's mouth and deforms to make precise contact with the user's teeth, the bristle-free operation can thus provide the required degree and uniformity of pressure required for cleaning the teeth and massaging of gums, for both the top and bottom rows of teeth. Moreover, the user can provide gentle biting pressure to operate the teeth cleaning device 100, and/or select one or more vibration patterns (e.g., slower/faster frequency and/or vibration pattern or cadence) of the teeth cleaning device 100, without having to clench the user's jaws or apply significant biting pressure. This significantly reduces the risk of damaging the user's teeth and/or jaws. The teeth cleaning device 100 provides several vibration patterns based on the gentle pressure exerted by the user, which can suit different conditions of the user's teeth and gums and keep the user's teeth and gums healthy.

Moreover, upon start and during a certain time thereafter, the vibration and dispensing assembly 202 is configured to receive the cleaning and whitening gel from the gel assembly 222, and further to dispense the gel for cleaning and/or whitening into the mouth guard 106, which then communicates the gel into the user's mouth through openings in the top of the mouth guard for the upper row of teeth and through openings in the bottom mouth guard for the bottom row of teeth. The openings in the mouth guard 106 to dispense the gel will be described hereinafter in greater detail with reference to FIGS. 3A and 3B.

The vibration and dispensing assembly 202 includes a housing base 204, an isolation layer 206, a housing cover 208, a bite force sensor (FSR) assembly 210, and a plurality of sonic vibration disks (motors) 212. The housing base 204 receives the isolation layer 206, the FSR assembly 210, and the vibration disks 212, while the housing cover 208 secures the foregoing components in the housing base 204 to complete the vibration and dispensing assembly 202. The isolation layer 206 is a sealing member that restricts any water, liquid, or other fluid material (e.g., saliva, gel, cleaning agent, etc.) from contacting the vibration disks 212 in the vibration and dispensing assembly 202.

The bite force sensor assembly 210 senses pressure provided by the user in biting the mouth guard 106. At initial activation of the pressure sensor 432 of the FSR assembly 210 described with reference to FIGS. 4A-4C, a certain amount of cleaning and whitening gel can be dispensed, e.g., for a certain time period at a certain flow rate, upon the initial activation of the pressure sensor 432 of the FSR assembly 210. At an interval of time after the initial activation of the sensor 432, the sonic vibration disks 212 are then configured to vibrate, e.g., sonically, and the vibration can be adjusted according to one or more selectable vibration patterns or cadence.

The light assembly 214 is configured to activate cold light, which can be transmitted into the user's oral cavity via the mouth guard 106. More specifically, at approximately the interval of time after the initial activation of the sensor 432, the light assembly 214 is configured to activate cold light. In this regard, the mouth guard 106 is clear, transparent, or translucent. The mouth guard 106 can thus allow the cold light emitted by the light assembly 214 to pass through the mouth guard 106 into the user's oral cavity, and further to activate a whitening (bleaching) compound in the cleaning and whitening gel that provides whitening of the teeth, e.g., thus allowing simultaneously to remove plaque on the teeth by activating vibration of the gel against the user's teeth, and to provide whitening of the teeth by activating the whitening compound in the gel via the cold light. More specifically, the cold light that is emitted by light assembly 214 activates the whitening compound in the gel using a blue light (e.g., wavelength between 480 nm and 520 nm), which has proven effective in whitening discolored teeth, as will be described hereinafter in greater detail.

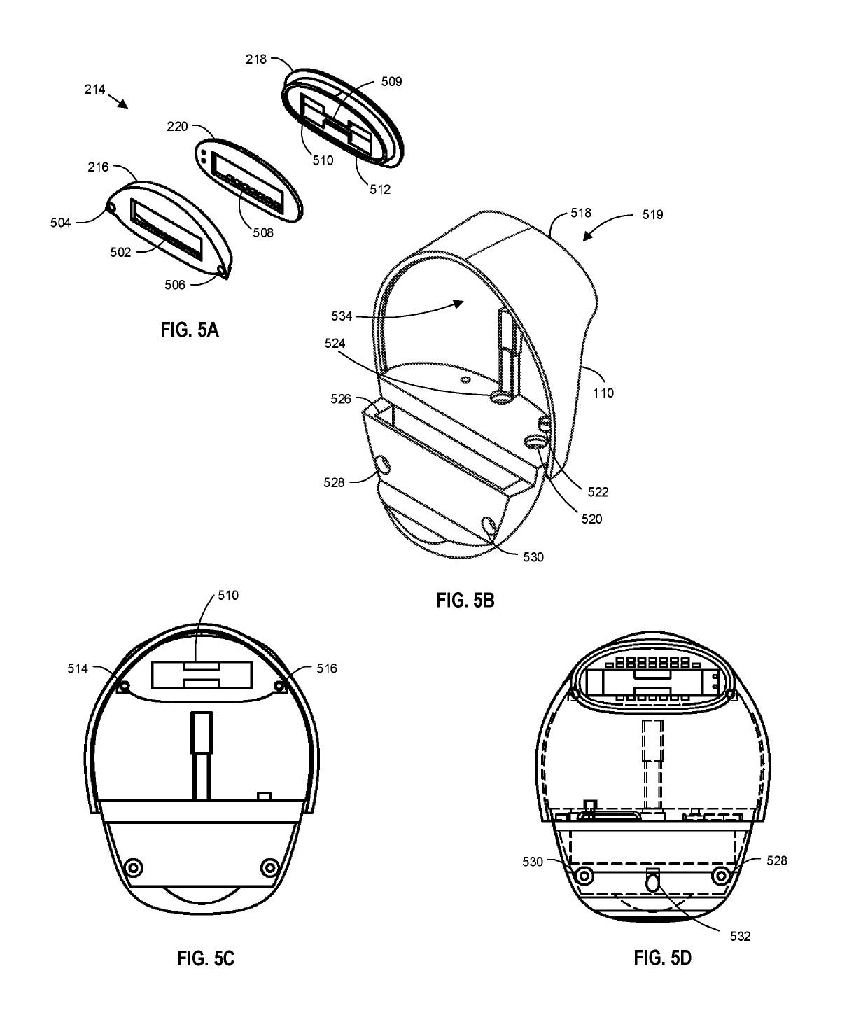

The light assembly 214 includes a housing base 216, a housing cap 218, and a cold light controller 220. The housing base 216 is secured to the shell section 110, such as via pins provided in the shell section 110 that are received through respective openings of the housing base 216, in order to secure the cold light controller 220 to the shell section 110 in a light-communicating relationship with reference to the housing cap 218. The housing cap 218 has a base and a body, wherein the body extends into the shell section 110. The base of the housing cap 218 can be sonically soldered or glued in relation to the opening 519 on the face 518 of the shell section 110, as illustrated in FIGS. 5D and 6D. As will be described hereinafter in greater detail, the housing cap 218 will engage the vibration and dispensing assembly 202.

The gel assembly 222 is configured to dispense the cleaning and whitening gel when the FSR assembly 210 senses an initial pressure provided by the user through the mouth guard 106. More specifically, a certain amount of gel can be dispensed upon start of operation of the teeth cleaning device 100. Similarly, the gel can also be dispensed upon a predetermined pattern of activation of the FSR assembly 210, e.g., certain pattern and/or length of time of the activation.

The gel assembly 222 includes housing members 224 and 226, an air pump 228, a gel cartridge 230, air tubes 232, and gel tubes 234. The housing members 224, 226 secure the air pump 228 via respective recesses in the shell section 110. Moreover, the housing member 224 and the shell section 110 also include respective recesses, which in combination secure the rechargeable battery 246 in shell assembly 108.

The gel cartridge 230 stores the cleaning and whitening gel that is configured to provide cleaning and whitening to the user's teeth. The air pump 228 is configured to pump air sufficient to dispense gel from the cartridge 230 and flow the gel to the mouth guard 106 for ultimately dispensing the gel into the user's oral cavity. More specifically, using the tubes 232, the air pump 228 intakes ambient air and outputs the air into the cartridge 230 to create a vacuum that pressurizes gel in the gel cartridge 230. As such, the gel flows from the cartridge through gel tubes 234 to the vibration and dispensing assembly 202, and is ultimately dispensed through the mouth guard 106 into the user's oral cavity.

The gel cartridge 230 is pre-filled with the cleaning-whitening gel that can be used for several cleaning/whitening cycles. After use, the gel cartridge 230 can be either disposable or refillable. If disposable, after the gel is used for several cleaning/whitening cycles, the cartridge 230 can removed from the teeth cleaning device 100 and disposed. In this case, the gel cartridge 230 is replaced with a new cartridge 230, e.g., pre-filled with gel, which can be easily installed into the teeth cleaning device 100. If refillable, the cartridge 230 can be removed from the teeth cleaning device 100, refilled with the cleaning-whitening gel, and then easily re-installed into the teeth cleaning device 100.

The charging assembly 236 is configured to recharge a rechargeable battery 246. In some embodiments, the charging assembly 236 includes inductive charging for charging the battery 246. Alternatively or in addition, the charging assembly 236 can also include a universal serial bus (USB) connector (not shown) to connect the charging assembly 236 to an electric source for recharging the battery 246.

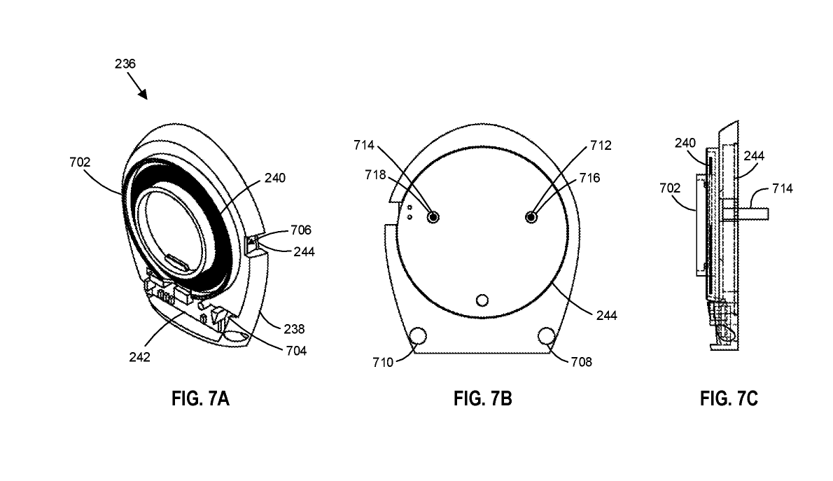

More specifically, the charging assembly 236 includes a housing 238, an induction coil 240, and an induction charging controller 242. The induction coil 240 is a ring that is disposed in a first recess of the housing 238, and the charging controller 242 is disposed in a second recess of the housing 238, which will be described in greater detail hereinafter with reference to FIGS. 7A-7C. The first and second recesses are located on a first side of the housing 238. The housing 238 also includes a third recess located on a second side of the housing in which the central controller 244 is disposed.

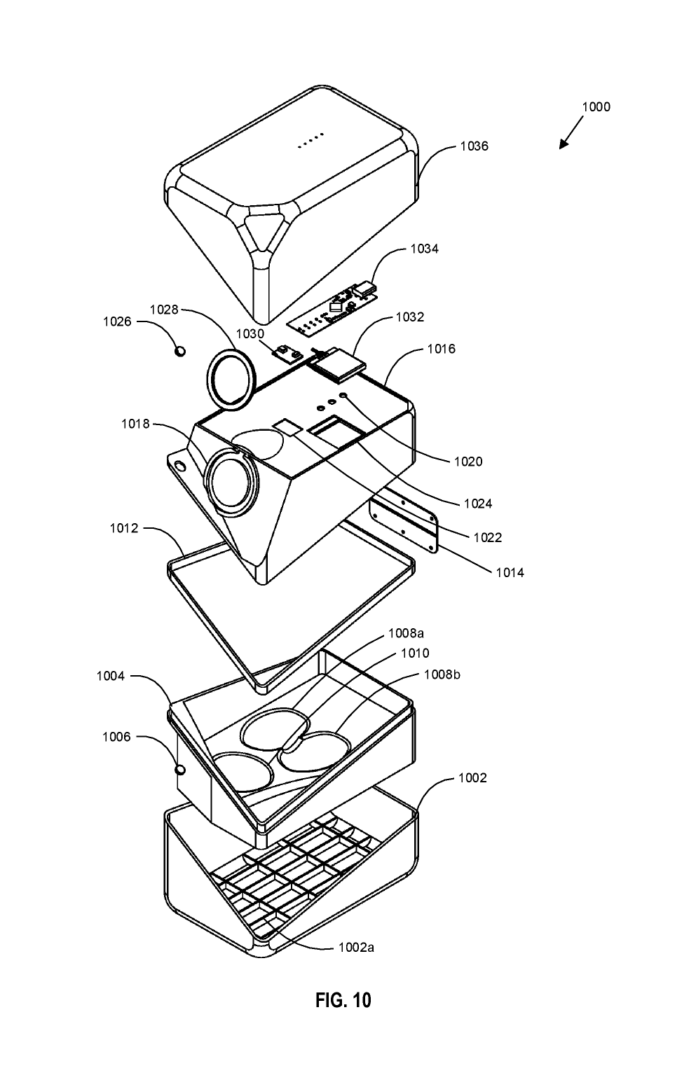

If the induction coil 240 of the charging assembly 236 is in proximity of a second induction coil 1028 of a charging/cleaning device 1000, which generates an electromagnetic field, then the charging controller 242 can charge the rechargeable battery 246 through the induction coil 240. In this regard, the example charging/cleaning device 1000 is described in greater detail hereinafter with reference to FIG. 10. However, if the charging assembly 236 is connected to an electric source via the USB connector as described hereinabove, the charging controller 242 can then charge the rechargeable battery 246 through the USB connector.

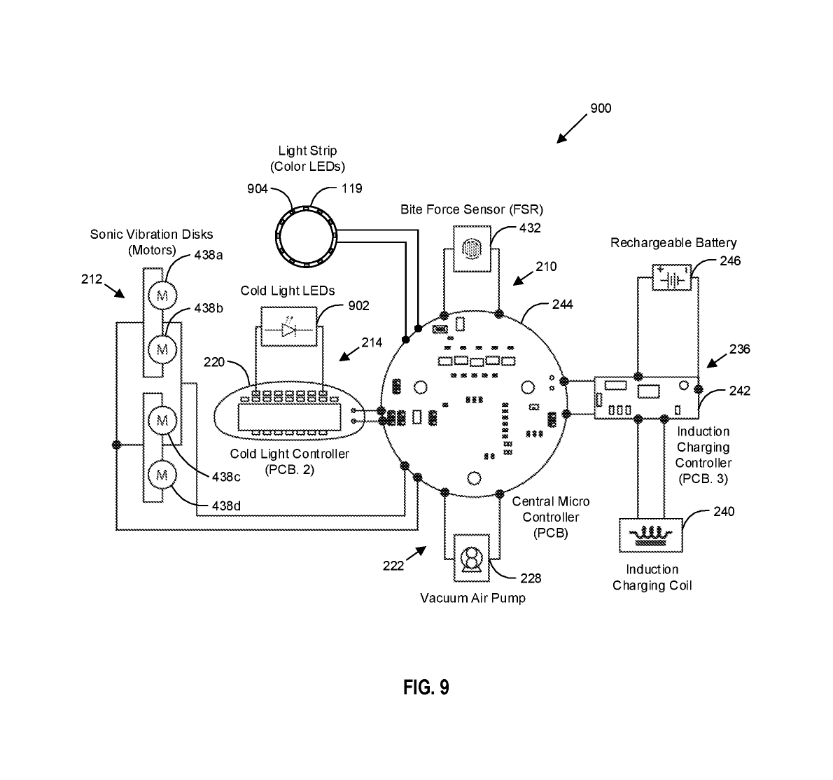

The central controller 244 is configured (or programmed) to control the functions of the teeth cleaning device 100. The central controller 244 can be a printed circuit board (PCB) or a programmed computing device. The cold light controller 220 and charge controller 242 are connected to the central controller 244, as described in greater detail hereinafter with reference to FIG. 9. The controllers 220, 242 can also be respective PCBs or programmed computing devices. Alternatively, the controllers 220, 242 can be incorporated into the central controller 244, which can thus be a PCB or a programmed computing device.

The rechargeable battery 246 is connected to the central controller 244 and is a main source of power to the teeth cleaning device 100. As described, the controllers 220, 242 are connected to the central controller 244, and thus controllers 220, 242 can receive power for their operation from the rechargeable battery 246 through the central controller 244.

The shell sections 110-118 of shell assembly 108 and the mouth guard 106 of the mouth assembly 102 thus receive the various electrical, mechanical, and replaceable and/or fungible components to form the teeth cleaning device 100, as illustrated in FIGS. 1A-1D.

FIGS. 3A and 3B illustrate an example mouth guard 106 illustrated in FIGS. 1 and 2.

As particularly illustrated in FIG. 3A, the mouth guard 106 includes a planar interface surface 302 and an opening 304. The LEDs of the light assembly 214 can illuminate with cold light the mouth guard 106 through the opening 518 of the shell section 110 and the interface surface 302 of the mouth guard 106. More particularly, the interface surface 302 mates with a reciprocal surface of the housing cap 218 in the light assembly 214, so that the LEDs of the light assembly 214 can illuminate with cold light the mouth guard 106 through the housing cap 218 in the opening 518 of the shell section 110 and also the interface surface 302 of the mouth guard 106, and can thus activate the whitening compound in the cleaning and whitening gel via the cold light in order to provide whitening of the user's teeth.

The opening 304 is configured to facilitate passage of a body portion 452 of the vibration and dispensing assembly 202, so that the surface of the body portion 452 formed by the base 204 and the cover 208 abuts the surface of the housing cap 218, and the gel guides 454, 456 are inserted into the reciprocal receiving openings 510, 512 of the opening 510 in the housing cap 218. The surface of body portion 452 and the gel guides can be glued into the housing cap 218, and in some cases the interface surface 302 of mouth guard 106 can also be glued to an interface surface of the housing cap 218. It should be noted that the body portion 452 and the gel guides 454, 456 are described in greater detail hereinbelow with reference to FIGS. 4A-4C.

As particularly illustrated in FIG. 3B, the mouth guard 106 is generally arcuate in shape, and includes left and right arm sections 306 and 310, respectively. Adjacent arcuate ridges in the top of the mouth guard 106 form the arcuate recess 104a in the top of the mouth guard that extends from the left arm section 306 to the right arm section 310. While not illustrated in FIG. 3B, similar adjacent arcuate ridges in the bottom of the mouth guard 106 form the arcuate recess 104b in the bottom of the mouth guard 106 that extends from the left arm section 306 to the right arm section 310. The arcuate recesses 104a, 104b receive the user's top and bottom rows of teeth and gums, respectively.

In the top of the mouth guard 106, the left and right arm sections 306, 310 include respective sets of openings 308, 312 that facilitate the dispensing of the cleaning and whitening gel from similarly shaped and located openings in the top of the vibration and dispensing assembly 202. While not illustrated in FIG. 3B, similar respective sets of openings in the bottom of the mouth guard 106 also facilitate the dispensing of the cleaning and whitening gel from similarly shaped and located openings in the bottom of the vibration and dispensing assembly 202.

It should be noted that the vibration and dispensing assembly 202 is disposed in a cavity 305 of the mouth guard 106 that is located between the arcuate recesses 104a, 104b. The cavity 305 is similarly shaped to the vibration and dispensing assembly 202 so as to securely and precisely receive the vibration and dispensing assembly 202 inside the mouth guard 106. For brevity and clarity of this description, the vibration and dispensing assembly 202 is not shown inside the mouth guard 106 in FIGS. 3A and 3B.

FIGS. 4A-4C illustrate an example vibration and dispensing assembly 202 illustrated in FIG. 2. As particularly described with reference to FIG. 2, the vibration and dispensing assembly 202 includes the housing base 204, the isolation layer 206, the housing cover 208, the FSR assembly 210, and the plurality of sonic vibration disks motors 212.

As illustrated in FIG. 4A, the housing base 204 is an arcuately shaped member that, when assembled with other components into the mouth assembly 102, fits into the mouth guard 106 and ultimately into the user's mouth. The housing base 204 includes a body 402 and arms 404, 412 that extend from the body 402 to form the arcuate housing base 204.

The left arm 404 includes openings 406, 408, 410, while the right arm 404 includes openings 414, 416, 418. The openings 406, 410 are for vibration disks 438a, 438b and opening 408 is a conduit for the cleaning and whitening gel, while the openings 414, 146 are for vibration disks 438c, 438d and the opening 416 is a conduit for the cleaning and whitening gel.

The isolation layer 206 is received into the housing base 204. As described hereinbefore with reference to FIG. 2, the isolation layer 206 is a sealing member that restricts any water, liquid, or other fluid material (e.g., saliva, gel, cleaning agent, etc.) from contacting the vibration disks 212 in the vibration and dispensing assembly 202. The isolation layer 206 is generally an arcuately shaped member that includes a body 420 and arms 424, 428 that extend from the body 420.

The arms 424, 428 of the isolation layer 206 include respective tabs 426, 430 at terminal ends of the arms 424, 428 that isolate or close the openings 408, 416 associated with dispensing cleaning and whitening gel from the openings 406, 410, 414, 418 associated with the respective vibrations disks 438a-438d. Moreover, the body 420 includes a recess 422 that receives a touch sensor 432 and a button 434 of the FSR assembly 210, while the button cover 436 is disposed atop of the body 420 over the touch sensor 432 and a button 434 disposed in the recess 422.

As illustrated in FIG. 4B, the isolation layer 206, the FSR assembly 210, and the plurality of vibration disks 212 are disposed in the housing base 204. The housing cover 208 includes a body 440 and arms 444, 448 that extend from the body 440 to form the arcuate housing cover 208. The housing cover 208 includes an opening 442 and openings 446, 450, and is disposed over the assembled housing base 204 as described immediately hereinabove. The openings 446, 450 are isolated over the openings 408, 414 in the respective arms 404, 412, associated with dispensing of the cleaning and whitening gel.

As illustrated in FIG. 4C, the opening 442 of the housing cover 208 is disposed over the button cover 436 of the FSR assembly 210, and the openings 446, 450 are disposed over the openings 408, 416. The housing cover 208 can be glued or sonically soldered to the housing base 204 in order to form the completed vibration and dispensing assembly 202.

While not shown for brevity and clarity, the housing base 204 also includes openings at the bottom of the arms 404, 412 that are similarly shaped and located to the openings 446, 450 in the housing cover 208, which openings are also similarly isolated under the respective openings 408, 414 in the arms 404, 412, associated with dispensing of the cleaning and whitening gel.

As further illustrated in FIG. 4C, the completed vibration and dispensing assembly 202 includes a body portion 452 and gel guides 454, 456. As described hereinabove with reference to FIG. 2, the surface of body portion 452 formed by the base 204 and the cover 208 abuts the surface of the housing cap 218, and the gel guides 454, 456 are inserted into the reciprocal receiving openings 510, 512 of the opening 509 in the housing cap 218, illustrated in FIG. 5A. The surface of body portion 452 and the gel guides 454, 456 are secured in the housing cap 218, e.g., via gluing or sonic soldering.

FIGS. 5A-5D illustrate a light assembly 214 and example integration of the light assembly 214 into the shell section 110 illustrated in FIG. 2.

The light assembly 214 includes a housing base 216, a housing cap 218, and a cold light controller 220. The housing base 216 includes a central opening 502 and openings 504, 506. The cold light controller 220 includes a central opening 508, and the housing cap 218 includes a central opening 509, which defines openings 510, 512.

As illustrated in FIGS. 5A and 5B, on a first side of the shell section 110, the shell section 110 includes a generally semi-circular or arcuate opening 534 terminating with a planar bottom, openings 520, 522 in the bottom for air tubes 602, 604, an opening 524 in the bottom for a gel tube 606, and an opening 526 in a section recessed below the bottom for the battery 246. Moreover, openings 528, 530 are provided in the recessed section for connection screws. On the second side of the shell section 110, the shell section 110 includes a face 518 at the top of the shell section 110, a generally oval opening 519 through the face 518, and an opening 532 in the recesses section for a connection post 616. It should be noted that the oval opening 519 is in communication with the semi-circular opening 534.

As illustrated in FIGS. 5C and 5D, the housing base 216 is secured to the shell section 110 via respective pins 514, 516 provided in the shell section 110 that are received through openings 504, 516 of the housing base 216, which also secures the cold light controller 220 to the shell section 110 in a light-conducting relationship with reference to the housing cap 218, with the mating of the respective openings 502, 508, 509, as illustrated in FIG. 5D.

As further illustrated in FIG. 5D, the housing cap 218 has a base and a body that extends into the shell section 110 through the opening 519 in the face 518 of the shell section 110, as illustrated in FIG. 6D. The base of the housing cap 218 can be sonically soldered or glued to the face 518 of the shell section 110.

Moreover, the housing cap 218 is clear, transparent, or translucent so as to allow the cold light emitted by the cold light device 220 to pass through the light assembly 214 into the mouth guard 106 and ultimately into the user's oral cavity, and further to activate a whitening (bleaching) compound in the cleaning and whitening gel that provides whitening of the teeth, e.g., thus allowing simultaneously to remove plaque on the teeth by activating vibration of the gel against the user's teeth, and to provide whitening of the teeth by activating the whitening compound in the gel via the cold light.

FIGS. 6A-6D illustrate a gel assembly 222 and example integration of the gel assembly 222 into the shell section 110 illustrated in FIG. 2.

The gel assembly 222 includes housing members 224, 226, an air pump 228, a gel cartridge 230, air tubes 232, and gel tubes 234. The housing members 224, 226 secure the air pump 228 via their respective recesses in the shell section 110. The housing members 224, 226 can snap fit together or can be glued. Moreover, the housing member 226 includes an opening 227 via which the housing members 224, 226, and ultimately, the air pump 228, can be secured to the shell section 110 using a screw (not shown), which can be received through the opening 624 into opening 227 of the housing member 226, illustrated in FIGS. 6A and 6C. Moreover, the housing member 224 includes a recess 622 and the shell section 110 includes an associated recess 526, which in combination can secure the rechargeable battery 246 in shell assembly 108.

The gel cartridge 230 of FIG. 6A is advanced along arrow 628 illustrated in FIG. 6D until the planar top surface of the gel cartridge 230 engages the reciprocal planar bottom surface of the shell section 110, with a guide post 616 engaging a reciprocal opening 532 in shell section 110, as illustrated in FIGS. 6A and 6D.

The air tubes 232 include tubes 602, 604. As particularly illustrated in FIGS. 6A-6D, the tube 602 engages the air output opening of the air pump 228 and the air input of the gel cartridge 230 through the opening 520 in the shell section 110, while the tube 604 engages an air vent 626 on the back of shell section 110 to draw in ambient air, which allows the air pump 228 to pressurize the gel in the gel cartridge 230. It should be noted that for the brevity and clarity of the description, the air pump 528 is shown in FIG. 6B without the housing members 224, 226.

The gel tubes 234 include tubes 606, 608, 610, and 612. The tube 606 is connected to the gel output 614 in the bottom of the back of the gel cartridge 230. Upon pressurization of the gel cartridge 230 using the air pump 228, the tube 606 conveys a stream of gel from the gel cartridge 230 to the t-connector tube 608, which splits the stream of gel into two streams of gel that are conveyed through the left and right tubes 610, 612 to the respective left and right gel guides 454, 456 of the vibration and dispensing assembly 202.

These aforementioned gel streams are dispensed through the respective openings 446, 450 in the vibration and dispensing assembly 202, which are in communication with the respective openings 308, 312 in the mouth guard 106, ultimately into the user's mouth. It should be noted that the openings to dispense the gel are provided in the top and bottom of the vibration and dispensing assembly 202, as well as in the top and bottom of the mouth guard 106. Accordingly, the cleaning and whitening gel is dispensed to the top and bottom teeth in the user's oral cavity.

FIGS. 7A-7C illustrate a charging assembly 236, a central controller 244, and their example integration into the shell section 110 illustrated in FIG. 2.

As illustrated in FIG. 7A, the charging assembly 236 includes a housing 238, an induction coil 240, and a charging controller 242. The induction coil 240 is a ring that is disposed in a first recess 702 of the housing 238, and the charging controller 242 is disposed in a second recess 704 of the housing 238, wherein the first and second recesses 702, 704 are located on a first side of the housing 238. The housing 238 also includes a third recess 706 on a second side of the housing 238 in which the central controller 244 is disposed.

As illustrated in FIGS. 7B and 7C, the housing 238 includes several posts 712, 714 extending from the second side of the housing 238. As further illustrated therein, the central controller 244 also includes reciprocal openings 716, 718, which are used in combination with the posts 712, 714 to precisely dispose the central controller 244 in the third recess 706 of the housing 238. Moreover, the posts 712, 714 are used to engage reciprocal openings 618, 620 in the air pump casing 224, so that the charging assembly 236 and the central controller 244 as disposed on the housing 238 can be integrated into the shell section 110.

FIG. 8 illustrates the teeth cleaning device 100 with the shell sections 112, 114, 116, the light strip 119, and the mouth guard 106 removed, thereby exposing the assembly and integration of the vibration and dispensing assembly 202, the light assembly 214, the gel assembly 222, the charging assembly 236, the central controller 244, and the rechargeable battery 246 into the shell section 110 of the teeth cleaning device 100 illustrated in FIGS. 1 and 2.

The vibration and dispensing assembly 202 can be integrated into the teeth cleaning device 100 by gluing as described before, or by another securing method. The light assembly 214, the gel assembly 222, the charging assembly 236, the central controller 244, and the rechargeable battery 246 are integrated into the shell section 110 of the teeth cleaning device 100, and secured therein using respective screws that extend through the openings of the housing 238 and engage the respective openings 528, 530 of the shell section 110, securing the aforementioned components in the teeth cleaning device 100.

FIG. 9 is a circuit diagram 900 that illustrates example electrical connections among the electrical and/or electronic components of the teeth cleaning device 100 illustrated in FIG. 1.

As shown in the circuit diagram 900, the cold light controller 220 and the induction charging controller 242 are connected to the central controller 244.

The cold light controller 220 of the light assembly 214 can include on its PCB a plurality of cold light emitting diodes (LEDs) 902, which are configured to emit a cold light (e.g., blue light having a wavelength between 480 nm and 520 nm). The LEDs 902 can be disposed on the PCB in one or more rows above and below the central opening 508, or in one or more other patterns about the cold light controller 220.

The rechargeable battery 246 and the induction charging coil 240 are connected to the induction charging controller 242 of the charging assembly 236. While not shown in the circuit diagram 900, the charging controller 242 can also include a USB connector for charging the recharging the battery 246 via another electrical source.

As further shown in the circuit diagram 900, the sonic vibration disks 438a-438d of the plurality of sonic vibration disks 212 included in the vibration and dispensing assembly 202 are connected directly to the central controller 244. While only four sonic vibration disks are illustrated, more or fewer sonic vibration disks can be provided in the vibration and dispensing assembly 202.

Moreover, the light strip 119 is also connected directly to the central controller 244. The light strip 119 includes a plurality of quad-color LEDs 904 for indication of one or more operational statuses of the teeth cleaning device 100. In this regard, the light strip 119 is clear, transparent, or translucent, and the LEDs 904 can be disposed on or embedded into the light strip 119 in one or more patterns about the light strip 119 to indicate the operational statuses.

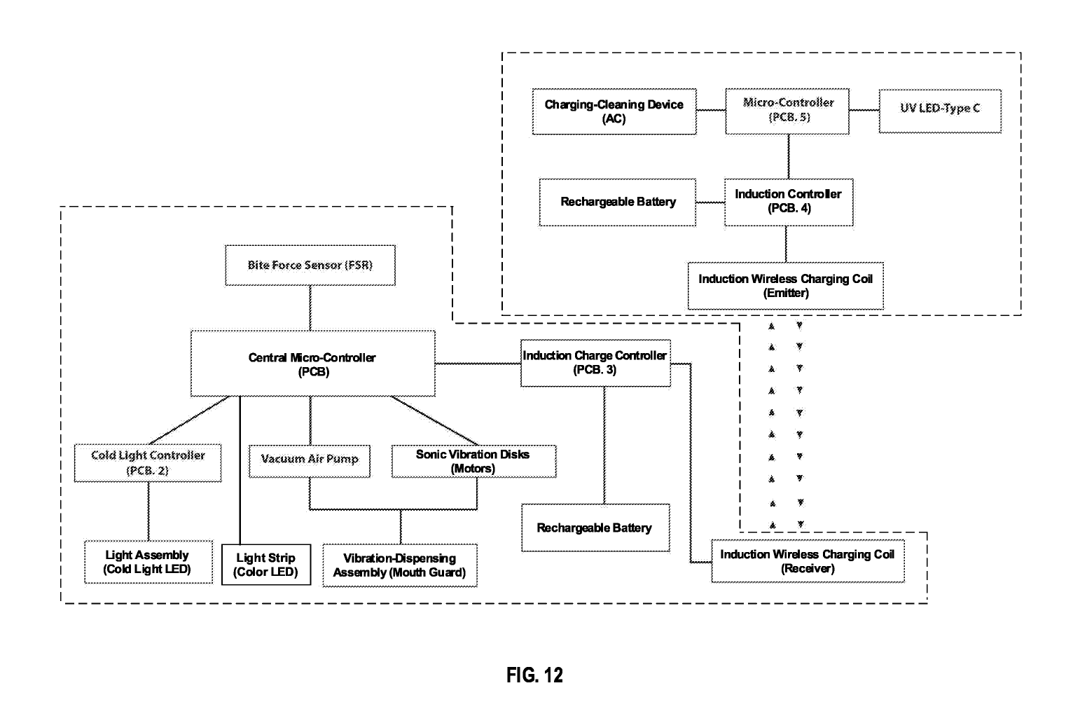

The pressure sensor 432 of the FSR assembly 210, and the air pump 228 of the gel assembly 222 are also connected directly to the central controller 244. While the circuit diagram 900 illustrates the electrical or electronic connections, the functional operations of the foregoing electrical and/or electronic components of the teeth cleaning device 100 will be described in greater detail hereinbelow with reference to FIG. 12.

FIG. 10 illustrates an example charging/cleaning device 1000 for the teeth cleaning device 100 illustrated in FIGS. 1-9.

The charging/cleaning device 1000 is configured to store, recharge, and sanitize the teeth cleaning device 100. The charging/cleaning device 1000 can be a travel device that is used to carry the teeth cleaning device 100, and which can also carry one or more replacement gel cartridges 1102, 1104.

The charging/cleaning device 1000 includes shell sections 1002, 1004, 1012, 1016, and 1036, and a hinge 1014. The shell section 1002 can include a lattice bottom 1002a. The shell section 1002 receives the shell section 1004, which is disposed on the lattice bottom 1002a.

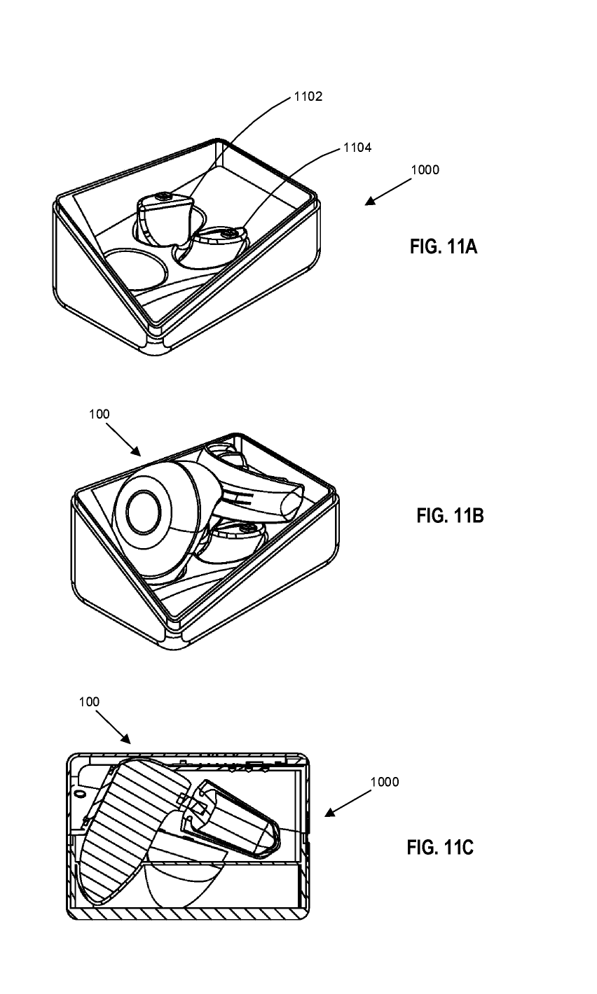

The shell section 1004 includes a magnet 1006, and openings 1008a, 1008b, and 1010. The hinge 1014 allows movement of top sections 1016 and 1036 with respect to the bottom sections 1002, 1004, and 1012 from a closed position to an open position, and vice versa. The magnet 1006 helps to secure the top sections 1016 and 1036 in closed position with respect to the bottom sections 1002, 1004, and 1012, using a reciprocal magnet 1026. The openings 1008a, 1008b are configured to receive and store the replacement gel cartridges 1102, 1104, as illustrated in FIG. 11A. The decor ring 1012 is a decorative component that is disposed about the shell section 1004.

The shell section 1016 includes recesses 1018, 1022, and 1024. The recess 1018 receives an induction coil (emitter) 1028 that can induce a charge of the rechargeable battery 246 in the teeth cleaning device 100 through the inducting coil (receiver) 240 of the charging assembly 236. The recess 1022 receives a wireless charging controller 1030, while the recess 1024 receives a rechargeable battery 1032, which can recharge and sanitize the teeth cleaning device 100 when charging/cleaning device 1000 is not connected to an AC source. The charging controller 1030 is responsible for charging the battery 1032 when the charging/cleaning device 1000 is connected to an AC power source, and also charging the battery 246 in the teeth cleaning device when the teeth cleaning device 100 is disposed in recess 1010. While no recess is shown for a central controller 1034, a similar recess in the shell section 1016 can be provided for the central controller 1034.

Moreover, the shell section 1016 includes UVC LEDs 1020 that can sanitize the teeth cleaning device 100, when the charging/cleaning device 1000 is closed. The foregoing electrical and electronic components are connected to the central controller 1034 in order to perform the various charge and/or sanitize regimens.

FIGS. 11A-11C illustrate several views of the example charging/cleaning device 1000 illustrated in FIG. 10 with the example teeth cleaning device 100 illustrated in FIGS. 1-9.

As particularly illustrated in FIG. 11A, the replacement gel cartridges 1002, 1004 can be disposed in the respective recesses 1108a, 1108b.

As further illustrated in FIG. 11B, the shell section 112 of the teeth cleaning device 100 can be disposed in the recess 1010, and the mouth guard 106 of the teeth cleaning device 100 can be disposed against the planar surface of shell section 1004, which is located after the recesses 1008a, 1008b.

As illustrated in FIG. 11C, the charging/cleaning device 1000 can sanitize the teeth cleaning device 100 and the replacement gel cartridges 1102, 1104, when the charging/cleaning device 1000 is closed.

FIG. 12 illustrates a logical diagram of the teeth cleaning device 100 illustrated in FIGS. 1-9, as well as the charging/cleaning device 1000 illustrated in FIGS. 10 and 11.

More specifically, in order to activate the teeth cleaning device 100, the user bites on the mouth guard 106, the biting pressure from which deforms the mouth guard 106, and in turn transfers the biting pressure to the bite force sensor 432 of the FSR assembly 210 in the vibration and dispensing assembly 202. More specifically, the deformation applies pressure to the button 434 and the button cover 436 of the FSR assembly 210, which in turn apply pressure to the force sensor 432. Upon sensing the pressure, the force sensor 432 sends a start signal to the central controller 244. The central controller 244 receives the start signal from the force sensor 432, and controls the other electrical and/or electronic components, e.g., including controllers 220, 242, in one or more cleaning and whitening run cycles based on the following programming.

If the controllers 220, 242 are incorporated into the central controller 244, then the central controller 244 directly controls the other electrical and/or electronic components, in the one or more cleaning and whitening run cycles based on the following programming.

Upon receiving the start signal, the central controller 244 controls the LEDs 904 of the light strip 119 to display a breathing pattern (e.g., rhythmical and repetitive on/off of the LEDs 904), e.g., in a white color, to indicate that the teeth cleaning device is working, or in other words, is in operational status. Moreover, the central controller 244 further controls the vacuum pump 228 to pressurize the gel cartridge 230 in order to dispense the cleaning and whitening gel. The gel flows from the cartridge 230 through gel tubes 234 to the vibration and dispensing assembly 202, wherein the gel is received in openings 408, 416 and dispensed through openings 446, 450, and ultimately the gel is dispensed through openings 308, 312 of the mouth guard 106 into the user's oral cavity. It should be noted that the foregoing openings are in the top and bottom, and therefore the cleaning and whitening gel is disposed to the top and bottom rows of the user's teeth.

After a predetermined duration (e.g., three seconds) from the receipt of the start signal, the central controller 244 controls the plurality of the sonic vibration disks 212, which delivers vibration to the user's oral cavity. This duration can be shorter or longer, but should generally be for a time period sufficient to dispense a predetermined amount of cleaning and whitening gel into the user's oral cavity. The mouth guard 106 deforms in the user's mouth from user's heat and biting, and thus fits precisely to structure and/or location of the user's teeth. The vibrations drive the cleaning and whitening gel (e.g., including a microscopic level of fine dental pumice and/or pearl powder) against the surfaces of the user's teeth in order to both clean and polish the teeth.

After approximately the same predetermined duration from the receipt of the start signal, the central controller 244 also sends a start signal to the cold light controller 220 that activates the cold light LEDs 902 which generate cold light, e.g., 480 nm to 520 nm, Ra>90, 3200k. The foregoing cold light activates whitening or bleaching compound (e.g., hydrogen peroxide) in the cleaning and whitening gel that de-carbonates plaques, stains, and/or pigmentation of the teeth.

During the run cycle, the force sensor 432 continues to sense user's bite pressure, e.g., every 10 ms, or at higher or lower interval of time, and sends the pressure signals to the central controller 244. Based on the pressure signals, the central controller 244 makes adjustments to the pattern and/or frequency of vibration delivered by the plurality of vibration disks 212. The adjustment to the pattern and/or frequency of vibration is relative to the biting pressure in real time, and is accordingly related to the cleaning intensity. More specifically, the harder the user bites, the more different the pattern and/or frequency of the vibration, which allow the user to reach the cleaning that the user desires.

A recommended default cleaning and whitening time of the run cycle is ten seconds (e.g., excluding initial three seconds to dispense gel). This predetermined time period can be extended. More specifically, the user can extend the time as the user desires. In this regard, the default time period is set to a default of ten seconds. At approximately ten seconds, the central controller 244 controls the plurality of vibration disks 212 to vibrate strongly three times, and also controls LEDs 904 of the light strip 119 to blink three times (e.g., in white or blue), indicating to the user that the run cycle is completing.

If the user would like to extend the default run cycle, the user can continue biting pressure on the mouth guard 106 to continue triggering the force sensor 432 in order to extend the run cycle. In this case, however, the central controller 244 does not run the vacuum pump 228 again, and accordingly no more gel is dispensed into the user's oral cavity. Moreover, if the user accidentally releases the mouth guard 106 during the default run cycle or the extended run cycle, the user can bite back on the mouth guard 106 within a predetermined time period (e.g., five seconds) in order to resume the default run cycle or the extended run cycle.

During the run cycle or the extended run cycle, the user can stop triggering the force sensor 432. In this case, the central controller 244 stops the operation of the sonic vibration disks 212, sends a signal to the cold light controller 220 to stop energizing the cold light LEDs 902, and stop energizing the LEDs 904 of light strip 119. However, the user can restart or continue the run cycle or the extended run cycle within the predetermined time period (e.g., five seconds) of stopping by again biting the mouth guard 106 and in turn triggering the force sensor 432. In this case, the central controller 244 again activates the operation of the sonic vibration disks 212, sends a signal to the cold light controller 220 to energize the cold light LEDs 902, and energizes the LEDs 904 of light strip 119 to provide a breathing indication. If the force sensor 432 does not sense pressure within the predetermined time of stopping the default run cycle or the extended run cycle, the central controller 244 resets the teeth cleaning and whitening device 100, which means that the central controller 244 will restart the overall cleaning and whitening run cycle, including the pump 228, the vibration disk 212, the cold light LEDs 902, and the color LEDs 904.

The central controller 244 can indicate different operational statuses of the teeth cleaning and whitening device 100. As already described above, the central controller 244 can control the LEDs 904 of the light strip 119 to display a breathing pattern that indicates that the teeth cleaning device is working. The central controller 244 can also indicate that the rechargeable battery 246 is full by a steady green color of the LEDs 904, or that the rechargeable battery 246 is low by a red blinking pattern of the LEDs 904. Other operational statuses can also be indicated by the central controller 244.

After the user finishes using the teeth-cleaning whitening device 100, the user simply rinses the device, e.g., using water, and then disposes the device 100 into the charging/cleaning device 1000. The charging/cleaning device 1000 can then inductively charge the battery 246 of device 100, as well as sanitize the device 100 via UVC LEDs 1020, as described in greater detail hereinbefore with reference to FIGS. 10-11C.

The above-described teeth cleaning device 100 facilitates cleaning and whitening of the user's teeth. The device 100 does not require manual manipulation, enabling the user to use the device 100 hands-free while engaging in other activities. The device 100 can provide further benefit to disabled users in using the device 100 without associated difficulties. Moreover, the device 100 combines sonic vibration and cold light in order to mechanically and chemically activate a cleaning and whitening gel for cleaning and whitening the user's teeth approximately simultaneously. The device 100 includes a mouth guard 106 formed as a two-sided tray for both top and bottom rows of teeth, as well as the gums of the user's oral cavity. The mouth guard 106 deforms about the teeth and the gums, and is vibrated to provide non-irritating frictional contact to the surfaces of the teeth as well as the soft tissues of the gums for cleaning. The mouth guard 106 further enables cold light to be transmitted through the mouth guard 106 into the user's oral cavity for whitening.

Thus, a bristle-less teeth cleaning device with automatic gel dispensing for combined mechanical and chemical activation has been described. Although specific example embodiments have been described, it will be evident that various modifications and changes may be made to these embodiments without departing from the broader spirit and scope of the invention.

Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments shown are described in sufficient detail to enable those skilled in the art to practice the teachings disclosed herein. Other embodiments may be utilized and derived therefrom, such that structural and logical substitutions and changes may be made without departing from the scope of this application.

The foregoing detailed description, therefore, is not to be taken in a limiting sense, and the scope of various embodiments is defined only by the appended claims, along with the full range of equivalents to which such claims are entitled.

Although specific embodiments have been shown and described herein, it should be appreciated that any arrangement calculated to achieve the same purpose may be substituted for the specific embodiments shown. This application is intended to cover any and all adaptations or variations of various embodiments. Combinations of the above embodiments and other embodiments not specifically described herein, will be apparent to those of skill in the art upon reviewing the above description.

The Abstract is provided to comply with 37 C.F.R. § 1.72(b) and will allow the reader to quickly ascertain the nature of the technical disclosure of this application. It is submitted with the understanding that it will not be used to interpret or limit the scope or meaning of the claims.

In the foregoing detailed description, various features may be grouped together in a single embodiment for the purpose of streamlining the disclosure of this application. This method of disclosure is not to be interpreted as reflecting that the claimed embodiments have more features than are expressly recited in each claim. Rather, as the following claims reflect, inventive subject matter lies in less than all features of a single disclosed embodiment.

Moreover, it is contemplated that the features or components of various embodiments described herein can be combined into different combinations that are not explicitly enumerated in the foregoing detailed description and that such combinations can similarly stand on their own as separate example embodiments that can be claimed.

1. A teeth cleaning device comprising:
a gel cartridge storing a cleaning and whitening gel;
a plurality of light emitting diodes configured to emit a cold light;
a mouth guard having a generally arcuate shape, the mouth guard comprising a first arcuate recess, a second arcuate recess opposite the first arcuate recess, a cavity, a first surface, and a plurality of first openings, the first arcuate recess located on a top side of the mouth guard to receive a first row of teeth, the second arcuate recess located on the bottom side of the mouth guard to receive a second row of teeth, the cavity located between the first recess and the second recess, the first surface in communication with the cold light of the light emitting diodes;
a vibration and dispensing assembly having a generally arcuate shape and being disposed in the cavity of the mouth guard between the first arcuate recess and the second arcuate recess, the assembly comprising a plurality of vibration motors and a plurality of second openings, the motors being configured to generate vibrations, the second openings being in communication with the first openings; and
a central controller configured to control:
dispensing the gel from the gel cartridge through the second openings and the first openings into a user's oral cavity;
emitting the cold light from the light emitting diodes through the mouth guard into the user's oral cavity to activate a whitening compound in the gel; and
vibrating the plurality vibration motors configured to drive the gel against surfaces of the first row of teeth and the second row of teeth.

2. The teeth cleaning device of claim 1, further comprising an outer shell having one or more removable shell sections, the outer shell configured to house the gel cartridge, the light emitting diodes, the vibration and dispensing assembly, and the central controller.

3. The teeth cleaning device of claim 1, wherein the outer shell comprises a main shell section having a cavity, the main shell section having a generally semi-circular opening terminating with a planar bottom surface on a first side, and a generally oval opening at a top of a second side, wherein the generally oval opening is in communication with the semi-circular opening.

4. The teeth cleaning device of claim 1, further comprising:
air tubes;
gel tubes; and
an air pump configured to pressurize the gel cartridge using air tubes, and dispense the gel from the gel cartridge using gel tubes.

5. The teeth cleaning device of claim 4, wherein the air pump is disposed in a main shell section and the gel tubes engage gel guides of the vibration and dispensing assembly through the oval opening.

6. The teeth cleaning device of claim 5, wherein the main shell section includes an air vent connected by a first air tube to an input of the air pump in order to receive ambient air.

7. The teeth cleaning device of claim 6, wherein a second air tube is connected from an output of the air pump to an input in the gel cartridge.

8. The teeth cleaning device of claim 7, wherein a gel tube is connected to an output of the cartridge.

9. The teeth cleaning device of claim 3, wherein the light emitting diodes are disposed in the main shell section and emit the cold light through the oval opening into the first surface of the mouth guard.

10. The teeth cleaning device of claim 1, further comprising a cold light controller configured to communicate with the central controller to control the emitting of the cold light from the cold light emitting diodes.

11. The teeth cleaning device of claim 10, wherein the cold light emitting diodes are disposed on the cold light controller.

12. The teeth cleaning device of claim 1, further comprising:
a rechargeable battery; and
a charging controller configured to provide power from the rechargeable battery to the central controller.

13. The teeth cleaning device of claim 1, further comprising:
an induction coil configured to generate power when in proximity to a second induction coil; and
a charging controller configured to charge the rechargeable battery.

14. The teeth cleaning device of claim 1, further comprising a plurality of color light emitting diodes, wherein the central controller is further configured to control the color generating light emitting diodes to emit a color light indicating at least one operational status.

15. The teeth cleaning device of claim 1, further comprising a light strip disposed flush between several shell sections of an outer shell, wherein the light strip comprises a plurality of color light emitting diodes disposed on or embedded in the light strip.

16. The teeth cleaning device of claim 15, wherein the light strip is transparent.

17. The teeth cleaning device of claim 1, wherein the mouth guard is transparent.

18. The teeth cleaning device of claim 1, wherein the vibration and dispensing assembly further comprises a sensor configured to detect pressure from a user's bite through the mouth guard.

19. The teeth cleaning device of claim 18, wherein the central controller is configured to control dispensing the gel, emitting the cold light, and vibrating the motors based on the pressure detected by the pressure sensor.

20. The teeth cleaning device of claim 18, wherein the central controller is configured to control dispensing the gel for a first predetermined period of time, then followed by emitting the cold light and vibrating the motors for a second predetermined period of time.

21. The teeth cleaning device of claim 20, wherein the first predetermined period of time is three seconds, and the second predetermined period of time is ten seconds.

22. The teeth cleaning device of claim 18, wherein the central controller adjusts a pattern or a frequency of vibrating the motors based on an amount of the pressure detected by the pressure sensor.
